(19) United States
(12) Patent Application Publication
(10) Pub. No.: US 2003/0168613 A1
(43) Pub. Date: Sep. 11, 2003

(54) Multi-layer optical interference filter deposited by using only one starting coating material

(75) Inventors: Cheng-Chung Lee, Tainan (TW); Jin-Cherng Hsu, Tainan (TW)

Correspondence Address: BRUCE H. TROXELL, Suite 1404, 5205 Leesburg Pike, Falls Church, VA, 22041, US

(73) Assignee: Cheng-Chung Lee and YCL OPTCOM CO., LTD.

(21) Appl. No.: 10/092,588

(22) Filed: Mar. 8, 2002

(51) Int. Cl. A61N 5/00; G21G 5/00

(52) U.S. Cl. 250/492.1

(57) ABSTRACT

A multi-layer optical interference filter deposited by using only one starting coating material includes the steps of (a) providing a substrate in a vacuum chamber for deposition, and using an indirect monitoring method to monitor the formation of the thickness of deposition; (b) providing a silicon material in the vacuum chamber, and then using an electron beam gun to perform an evaporation treatment, producing silicon atoms for deposition; (c) guiding a gas mixture into the vacuum chamber to produce free ions for ion assisted deposition (IAD) and for combing with silicon atoms, and regulating the deposition rate to establish deposition parameters; and (d) using the deposition parameters thus obtained to establish a refractive index database, and then deriving the desired deposition parameters subject to the desired refractive index for deposition, so as to deposit the substrate into the desired multi-layer optical interference film product.

### BACKGROUND OF THE INVENTION

1. Field of the Invention

The present invention relates to a method of fabricating multi-layer optical interference films and, more particularly, to a multi-layer optical interference film fabrication method using single silicon material for deposition to produce multi-layer optical interference film products suitable for use in photoelectric display industry, optical communication industry, optical measuring instruments, energy control instruments, interference instruments, army weapons, and etc.

2. Description of the Related Art

Optical deposition is an importance technique in photoelectric technology. Currently, optical thin films are commonly used in treating optical signal and changing optical characteristics. When a photoelectric signal passed through an optically deposited element, the signal sensitivity is improved. Regular optical deposition designs are commonly of multi-layer optic interference films. According to conventional techniques, the formation of a multi-layer optic interference film is achieved by means of at least two materials. Because of being not able to find a material of the best-fit refractive index, two or more materials are used for co-evaporation. These conventional techniques are complicated and difficult to perform. Further, the finished products made according to the conventional techniques are less stable.

### SUMMARY OF THE INVENTION

The present invention has been accomplished to provide a multi-layer optical interference film fabrication method, which eliminates the aforesaid drawbacks. The invention uses silicon as prime evaporation material, an electron beam gun to produce silicon atoms, and an ion source to product mixed gas ions for forming the desired deposition. Therefore, a single silicon material can be evaporated into the desired multi-layer optic interference film product. During the fabrication process, single silicon material is used as prime evaporation material, and the composition of the gas mixture of nitrogen (N₂) and argon (Ar), or, oxygen (O₂) and argon (Ar) is regulated to achieve multiple layers of deposition of different refractive index.

### BRIEF DESCRIPTION OF THE DRAWINGS

FIG. 1 is a flow chart explaining a multi-layer optic interference film fabrication method according to the present invention.

FIG. 2 is a schematic drawing showing the fabrication of a multi-layer optic interference film according to the present invention.

### DETAILED DESCRIPTION OF THE PREFERRED EMBODIMENT

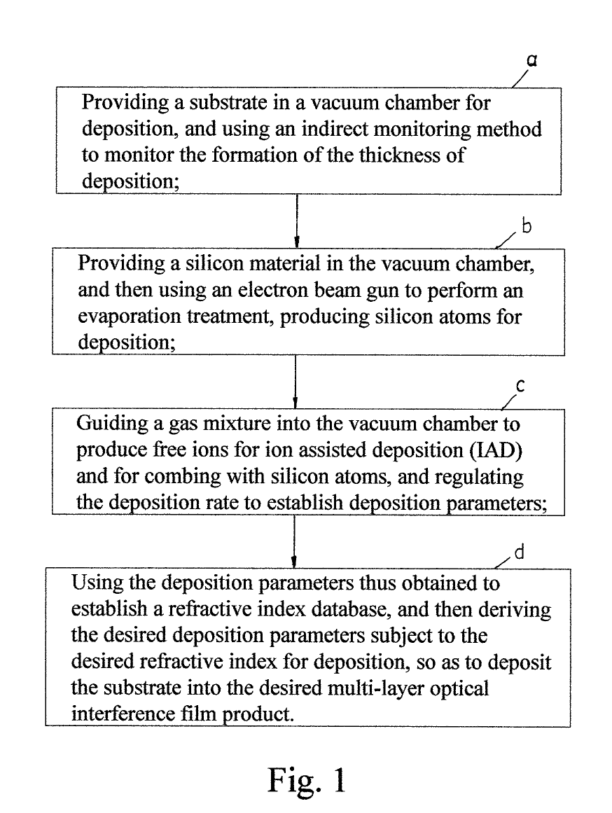

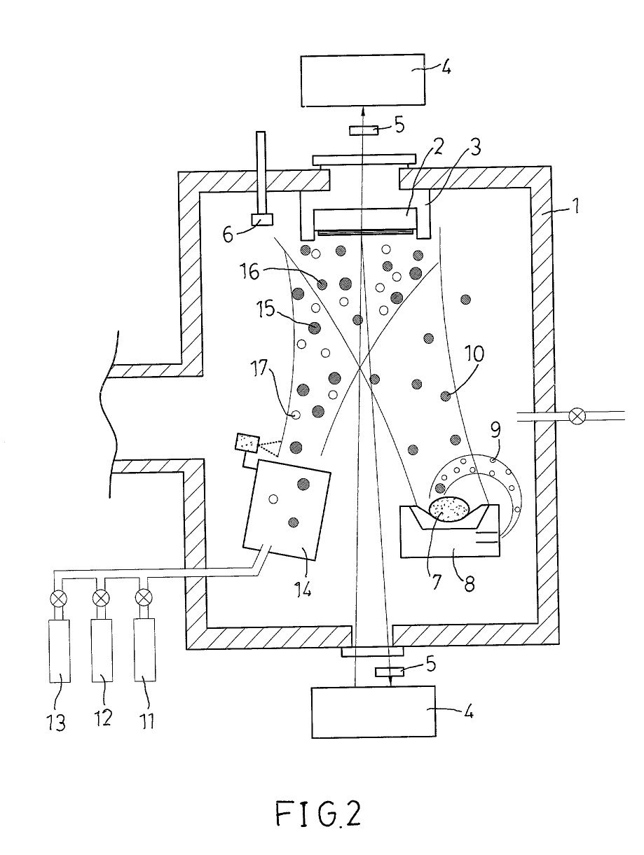

Referring to FIGS. 1 and 2, a multi-layer optical interference film fabrication method in accordance with the present invention comprises the steps of:

(a) providing a substrate in a vacuum chamber for deposition, and using an indirect monitoring method to monitor the formation of the thickness of deposition;

(b) providing a silicon material in the vacuum chamber, and then using an electron beam gun to perform an evaporation treatment, producing silicon atoms for deposition;

(c) guiding a gas mixture into the vacuum chamber to produce free ions for ion assisted deposition (IAD) and for combing with silicon atoms into a coating deposited on the substrate, and at the same time regulating the deposition rate to establish deposition parameters;

(d) using the deposition parameters thus obtained to establish a refractive index database, and then deriving the desired deposition parameters subject to the desired refractive index, so as to deposit the substrate into the desired multi-layer optical interference film product.

In the aforesaid step (a), as shown in FIG. 2, the substrate 2 is put in a substrate holder 3 in the vacuum chamber 1 for deposition. The deposition temperature is controlled within 100° C.˜250° C. The formation of deposition thickness is monitored by an indirect monitoring method. The indirect monitoring method uses two optical monitors 4 to monitor the intensity of optical interference signal produced from two monitoring filters 5 at two sides of the vacuum chamber 1 so as to monitor the optical thickness required for the formation of the desired deposition thickness, and a quartz monitor 6 in the vacuum chamber 1 to monitor the formation of deposition on the substrate 2. The optical thickness is the product of refractive index and deposition thickness.

In the aforesaid step (b), 99.999% pure silicon is used as original evaporation material. The selected silicon material 7 is put in the electron beam gun 8 inside the vacuum chamber 1 for evaporation. The electrons 9 emitted from the electron beam gun 8 triggers the silicon material 7 to produce free silicon atoms 10.

In the aforesaid step (c), nitrogen gas 11 and argon gas 13, or, oxygen gas 12 and argon gas 13 are guided into an ion source 14 in the vacuum chamber 1 to produce mixed gas ions of free nitrogen ions (N₂⁺) 15 and argon ions (Ar⁺) 17, or, oxygen ions (O₂⁺) 16 and argon ions (Ar⁺) 17. The mixed gas ions are then combined with free silicon atoms 10, forming a silicon nitride or silicon dioxide deposition on the substrate 2. The formation of the silicon nitride or silicon dioxide deposition can be controlled by changing the composition of the mixed gas ions produced by the ion source 14 subject to the desired refractive index. The refractive index of silicon nitride deposition is about within 1.45˜3.5 at wavelength 1550 nm. Because the refractive index is known, the ion source 14 can easily be adjusted to control the composition of the mixed gas ions, producing a silicon deposition having the desired refractive index. Further, the composition and flow rate of mixed gas in the vacuum chamber 1 affect the air pressure of the chamber and the ion current density of the mixed gas ions. Normally, the flow rate of the mixed gas is controlled within 0 sccm˜50 sccm, keeping the reactive gas pressure in the vacuum chamber 1 at the level that the vacuum around the substrate 2 is below 7×10⁻² Pa, preventing the formation of defective deposition.

Further, ion beam voltage, ion beam current, and material temperature in the ion source 14 affect the deposition quality. Normally, the density of ion current is controlled within 10 μA/cm²˜1 mA/cm², or preferably within 30 μA/cm²˜50 μA/cm²; ion beam voltage is controlled within 150V˜1000V.

According to tests made subject to the aforesaid control parameters, a deposition of refractive index at 3.5 is obtained when the density of nitrogen ion current controlled at 0 μA/cm²; a deposition of refractive index at 3.0 is obtained when the density of nitrogen ion current controlled at 10 μA/cm²; a deposition of refractive index at 2.0 is obtained when the density of nitrogen ion current controlled at 30 μA/cm²; a deposition of refractive index at 1.75 is obtained when the density of nitrogen ion current controlled at 40 μA/cm². Therefore deposition parameters can easily be established. Another deposition parameters can also be established by means of controlling the density of oxygen ion current.

In the aforesaid step (d), the function of the deposition parameters obtained in step (c) is stored in a computer for making a refractive index database, so as to obtain the desired deposition parameters subject to the desired refractive index. Subject to the light filtration effect of the desired product, the desired deposition parameters can be obtained by means of the operation of a software in the computer to run the deposition process, and to deposit coatings on the substrate, forming the desired multi-layer optic interference film.

For example, in the design of a multi-layer optical interference film of Si|M(HL)³H0.5L|Air, Si (silicon substrate) and Air represent the media at the left and right sides; “|” represents one each at the left and right sides of the interface; M, H, L, represent materials of different refractive index whose optical thickness is a deposition of ¼ wavelength; “³” represents three layers of HL deposition. Thus, the desired deposition parameters can easily be obtained subject to the desired refractive index, for producing the desired multi-layer optic interference silicon film product.

The invention uses silicon as prime evaporation material, an electron beam gun to produce silicon atoms, and an ion source to product mixed gas ions for forming the desired deposition. Therefore, a single silicon material can be evaporated into the desired multi-layer optic interference film product.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A multi-layer optical interference filter deposited by using only one starting coating material comprising the steps of:
(a) providing a substrate in a vacuum chamber for deposition, and using an indirect monitoring method to monitor the formation of the thickness of deposition;
(b) providing a silicon material in the vacuum chamber, and then using an electron beam gun to perform an evaporation treatment, producing silicon atoms for deposition;
(c) guiding a gas mixture into the vacuum chamber to produce free ions for ion assisted deposition (IAD) and for combing with silicon atoms, and regulating the deposition rate to establish deposition parameters; and
(d) using the deposition parameters thus obtained to establish a refractive index database, and then deriving the desired deposition parameters subject to the desired refractive index for deposition, so as to deposit the substrate into the desired multi-layer optic interference film product.

2. The multi-layer optical interference filter deposited by using only one starting coating material as claimed in claim 1, wherein the temperature of said substrate is controlled within 100° C.˜250° C. during step (a).

3. The multi-layer optical interference filter deposited by using only one starting coating material as claimed in claim 1, wherein said indirect monitoring method uses two optical monitor to monitor the intensity of optical interference signal produced from two monitoring filters at two sides of said vacuum chamber so as to monitor the optical thickness required for the formation of the desired deposition thickness, which is the product of refractive index and deposition thickness, and a quartz monitor in said vacuum chamber to monitor the formation of deposition on said substrate.

4. The multi-layer optical interference filter deposited by using only one starting coating material as claimed in claim 1, wherein the silicon material used in step (b) is 99.999% pure silicon.

5. The multi-layer optical interference filter deposited by using only one starting coating material as claimed in claim 1, wherein said gas mixture used in step (c) contains nitrogen and argon.

6. The multi-layer optical interference filter deposited by using only one starting coating material as claimed in claim 1, wherein said gas mixture used in step (c) contains oxygen and argon.

7. The multi-layer optical interference filter deposited by using only one starting coating material as claimed in claim 1, wherein the flow rate of said gas mixture is controlled within 0 sccm˜50 sccm, and the reactive gas pressure in said vacuum chamber is controlled within the level that the vacuum around said substrate is below 7×10⁻² Pa.

8. The multi-layer optical interference filter deposited by using only one starting coating material as claimed in claim 1, wherein the deposition rate during step (d) is controlled by regulating the composition of said gas mixture at said ion source and the reactive gas pressure in said vacuum chamber.

9. The multi-layer optical interference filter deposited by using only one starting coating material as claimed in claim 1, wherein the density of ion current produced by said ion source is controlled within 10 μA/cm²˜1 mA/cm², or preferably within 30 μA/cm²˜50 μA/cm²; and the ion beam voltage applied to said electron beam gun is controlled within 150V˜1000V.
